(19) United States
(12) Patent Application Publication
(10) Pub. No.: US 2010/0117842 A1
(43) Pub. Date: May 13, 2010

(54) Contaminant Detection Unit

(75) Inventor: Michail Fachidis, Neo Rysio (GR)

Correspondence Address: WARD AND SMITH, P.A., 1001 COLLEGE COURT, P.O. BOX 867, NEW BERN, NC, 28563-0867, US

(73) Assignee: WARNING, SA, Neo Rysio (GR)

(21) Appl. No.: 12/618,146

(22) Filed: Nov. 13, 2009

Related U.S. Application Data
(60) Provisional application No. 61/114,248, filed on Nov. 13, 2008.

(51) Int. Cl. G08B 21/00; G01N 33/22; B01D 35/143

(52) U.S. Cl. 340/618; 210/85

(57) ABSTRACT

Apparatuses and methods are provided for detecting contaminant in a fuel system, such as a fuel system of an internal combustion engine. The apparatus includes a fuel filter which includes an upper unit having an inlet port and an outlet port, and a closeable vent. A lower unit is releasably mounted to the upper unit and includes a contaminant collection zone and a contaminant detection sensor made up of a probe and a board with a microprocessor for detecting contaminant collecting in the collection zone within the lower unit. At least one indicator is provided for producing an alarm condition in a response to receiving a signal from the sensor.

### CROSS REFERENCE TO RELATED APPLICATIONS

This application is related to Provisional Application Ser. No. 61/114,248, filed Nov. 13, 2008, entitled Water-Detecting Unit with Self-Test Feature, to which priority is claimed, and the disclosure of which is specifically incorporated by reference herein.

### FIELD OF THE INVENTION

The invention relates generally to fuel systems and more particularly to providing apparatuses and methods for detecting and removing contaminants in a fuel system of an internal combustion engine.

### BACKGROUND ART

The fuel for use in internal combustion engines and heating systems is usually filtered before being fed to the engine or heating system. Filters are necessary because the presence of contaminants, such as water, in fuel systems has been found to be particularly disadvantageous for running efficiency of the machine that is using the fuel. For instance, as in the case with diesel or gasoline engines in boats or land vehicles, the presence of water in the fuel may cause corrosion of the fuel injector system with consequent reduction of the efficiency thereof and eventual complete breakdown thereof. Additionally, water in fuel systems may present a dangerous situation when power from an engine is necessary and water in the fuel results in the inability to deliver power at a crucial point in the operation of the engine.

In the past, various types of conventional fuel filters have been used in order to separate and/or remove water from the fuel prior to feeding the engine. These conventional fuel filters typically include a casing made of glass or steel that is mounted underneath a bracket having a fuel inlet and a fuel outlet and a filter element that is accommodated within the casing. The fuel introduced from the fuel inlet flows down a central passageway and enters the casing through a lower opening. The fuel changes flow direction to ascend in the casing and is filtered by the filter element and sent to the engine through the fuel outlet. In these conventional fuel filters, a major part of the water content included in the fuel may fall by gravity when the fuel flows from the opening. When the fuel is reversed in direction to flow upwardly, the remainder of the water content may attach to the surface of the filter element and accumulates to form droplets, which fall and gather in the bottom of the casing. A very small amount of water may pass through the filter element but such a small amount of water is very easily fully burned in the engine and typically does not affect engine efficiency. The water gathering in the bottom part of the casing may be removed at a suitable maintenance interval.

Drawbacks have been found in such conventional fuel filters. For instance, since a large amount of water may attach to the surface of the filter element and accumulate thereon when the filter is in use, the surface of the filter element applied with water gathering treatment or water repellant treatment is gradually attacked by the water over a period of time so that the life of the filter element is shortened. Furthermore, as a recent requirement especially related to exhaust gas purification, it is necessary to remove the water in the fuel to substantially zero in order to accurately maintain the measuring of the fuel amount at the fuel injection valve. From this requirement, it is necessary to separate the water content in the fuel as completely as possible before reaching the filter element.

An additional disadvantage to conventional fuel filters has been realized in regards to the water content accumulated in the bottom part of the casing of the fuel filter that has had to be manually removed at certain regular intervals. It has been found that if the water removing process is forgotten, the water level may reach the lower surface of the filter element wherein not only does the filter efficiency deteriorate but a large amount of water may be sent to the engine together with the fuel. This has an especially detrimental effect when the filter element is old and the filtering function has deteriorated wherein the water sent to the engine may cause rust to be introduced into the fuel injection system or may lead to other major efficiency decreases in engine production.

Due to the drawbacks of conventional fuel filtering systems, various apparatuses have been designed to attempt to resolve those negative characteristics by alerting the user of the presence of water and/or automatically draining collected water from the fuel system.

U.S. Pat. No. 4,276,161 to Matsui et al. discloses a fuel filter with a water level detector. A sensor responds to water level in the casing of the filter so that if the water level in the casing reaches a predetermined level, a low resistance condition between the electrode in the casing is detected by a water level detecting circuit. U.S. Pat. No. 4,562,431 to Jahnke et al. discloses a sensor system which is passive and which can only be used with one type of fuel system.

In a more recent development as described in U.S. Pat. No. 7,368,060 to Fachidis, the inventor in the present application, apparatuses and methods are disclosed for detecting contaminants in a fuel system. The apparatus described therein includes a programmable low voltage variable contaminant detection sensor formed integrally within an upper unit of a fuel filter unit, with probes extending into a lower unit of the fuel filter for sensing contaminant levels within a contaminant collection zone. Due to the fact that the sensor unit is programmable, the unit can be used in a variety of different types of fuel systems and overcomes some of the aforementioned disadvantages of the prior art.

In accordance with the invention described herein, there is provided an improved apparatus and method over that described in the references above, which are incorporated herein by reference. The present invention maintains the flexibility to be used in different fuel systems due to programmability, but also allows replacement of the sensor module thereof without requiring replacement of the entire fuel filter unit. The improved apparatus and method also allows for more usability by allowing the sensor module to be used with varying off-the-shelf contaminant filters.

### BRIEF SUMMARY OF THE INVENTION

In one aspect, the invention is an apparatus for detecting contaminants, e.g., water, in a fuel system. A fuel filter unit includes an upper unit having an upper section with a fuel inlet port, a fuel outlet port, and a closeable vent port. The upper unit includes a lower section with an open interior space bounded by walls thereof for receiving a fuel filter which is housed therein. The upper unit also includes an opening at the bottom thereof.

A lower unit is provided which is releasably mounted to the upper unit at the opening at the bottom, for sealing the upper unit. The lower unit has a programmable contaminant detection sensor which includes at least one detection probe extending upwardly into a fuel compartment region, for generation of a signal upon contact with a predetermined level of contaminant. A control board is housed within the lower unit and connected to at least one detection probe, and both make up the sensor. The control board includes a microprocessor and is capable of varying the predetermined level. An indicator is provided for generating an alarm condition in response to receiving the signal.

By having the sensor housed in the lower unit which is releasably mounted to the upper unit, it becomes possible to easily replace the sensor unit in the event of failure. Further, as noted, the sensor is programmable having a board with a microprocessor therein, and housed within the lower unit. In addition, due to the relative density differences between fuel and water, water tends to collect towards the bottom and is more easily detectable by the sensor resulting in an earlier warning, depending on the programming set for the unit, for fuel being fed to an internal combustion engine.

In an alternative aspect, there is provided a method of detecting contaminant in a fuel system of an internal combustion engine. The aforementioned contaminant detection apparatus is provided. The apparatus is then placed between the fuel source and the internal combustion engine. A microprocessor on the board is programmed with a predetermined level of contaminant selected for generating an alarm signal. Fuel is caused to enter the contaminant detection apparatus from the fuel source and the fuel is monitored so that an alarm signal is generated when the predetermined level of contaminant is reached. Preferably, the indicator for generating an alarm signal is one of a visual and audible signal so that two ways of perceiving the signal are provided.

### BRIEF DESCRIPTION OF THE DRAWINGS

FIG. 1 is a perspective view of the overall fuel system of an engine incorporating the contaminant detection apparatus provided in accordance with the subject matter disclosed herein.

FIG. 2 is a perspective partially exploded view of an embodiment of a contaminant detection apparatus provided in accordance with the subject matter disclosed herein.

FIG. 3 is a partial cross-section view of the lower unit of the contaminant detection apparatus illustrated in FIG. 2.

FIG. 4 illustrates a standard filter unit in accordance with the prior art having a releasable top and lower bowl.

FIG. 5 is a partial view of an alternative embodiment of the invention.

### DETAILED DESCRIPTION OF THE INVENTION

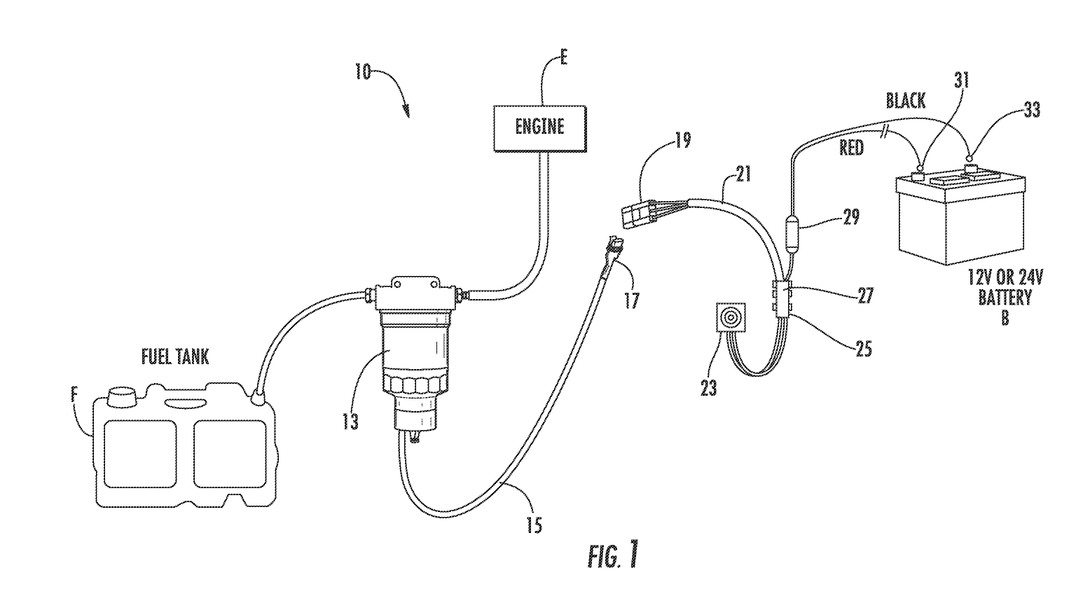

Referring now to FIG. 1, a fuel system incorporating the preferred embodiment of the contaminant detection apparatus of the present invention is shown. The fuel system includes a fuel source F that supplies fuel to engine E for the powering of an attached machine, wherein the fuel supplied by fuel source F may consist of gasoline, diesel or any other type of petroleum product. It is understood that engine E may comprise an internal combustion engine for powering a vehicle such as a marine craft or land vehicle, may comprise a burner system for heating applications, or may comprise any other application that utilizes petroleum products. A preferred embodiment of an apparatus for detecting contaminant in the fuel being fed from fuel source F is shown generally as 10, and is positioned in operative connection between fuel source F and engine E. The placement of contaminant detection apparatus 10 allows for detection and/or removal of contaminant in the fuel prior to the contaminated fuel reaching engine E. With such placement, the contaminant destruction of other fuel system parts of engine E, such as the injection system and the like, is minimized. While the most often cited contaminant in fuel systems is water, it is understood that the present subject matter may be directed to detecting any other contaminants found in fuels.

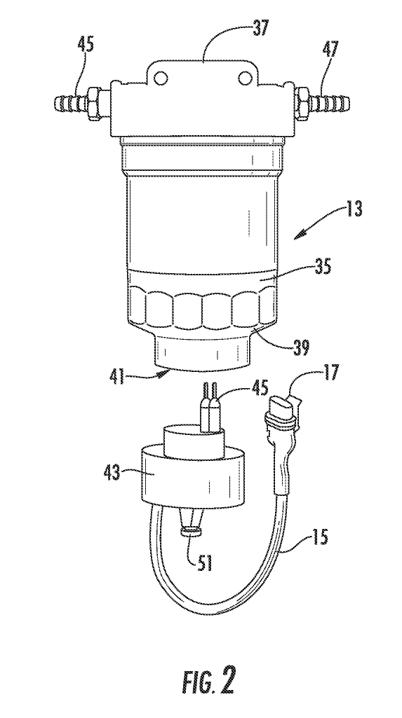

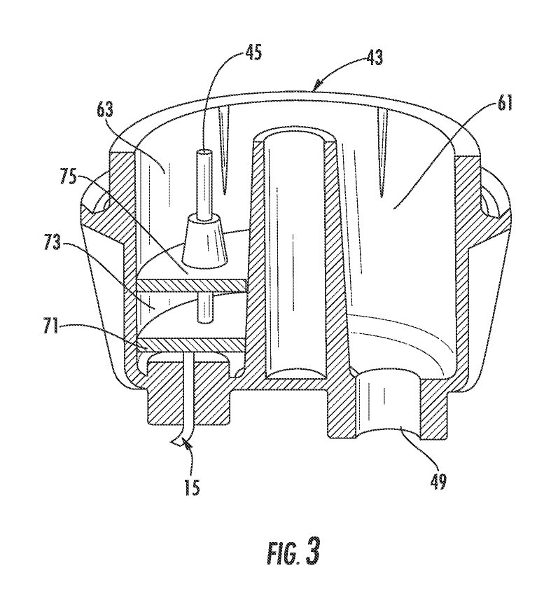

With reference to FIG. 1, and with further reference to FIGS. 2 and 3, contaminant detection apparatus 10 can include a fuel filter unit 13. The fuel filter unit 13 includes an upper unit 35 and a lower unit 43 and houses a fuel filter element (not shown) of any type known to those of skill in the art.

Upper unit 35 is preferably constructed of a metal material and includes an upper portion 37, including a fuel inlet port 45 for receiving of fuel from fuel source F and a fuel outlet port 47 wherein the fuel exits the fuel filter unit 13 and is fed to engine E. Upper portion 37 may also include a closeable vent port (not shown) for releasing vacuum within fuel filter unit 13 during draining of contaminant from the unit. The upper portion 37 which includes the inlet 45 and outlet 47, may be made detachable to allow replacement of a filter within the upper unit 35. The upper unit 35 also includes a lower portion 39 with an opening 41 for receiving a lower unit 43 therein.

As shown in FIGS. 2 and 3, the lower unit 43 includes one or more detection probes 45 that extend into the upper unit 35 through lower section 39 for sensing contaminant levels within a contaminant collection zone.

The contaminant detection sensor 45 measures the resistance variation in the fuel caused by the presence of water or other contaminants and is capable of determining when the resistance moves below a certain predetermined threshold level. The sensor probes 45 are continuously under voltage, with the voltage typically being a low-voltage such as 5 volts that will not cause sparking within the fuel source. The sensor probe 45 typically includes a control board 71 which may be housed in an isolated compartment 73 shown in FIG. 3, and incorporating a software program detection algorithm which is capable of varying levels of programmability. As such, the same contaminant detection probe 45 which is housed above an upper cap 75 of the isolated compartment 73 within which the control board 71 is housed (making up a sensor), may be used for different size engines where the capacity of the fuel filter unit can differ significantly.

The contaminant detection probe 45 is constantly monitoring fuel flowing through the fuel filter unit 13, and is adapted to generate a signal, such as an alarm signal, upon detection of a predetermined level of contaminant such as water in the contaminant collection zone.

As shown in FIG. 1, the overall contaminant detection apparatus 10 may further include at least one indicator 23. The indicator 23 and other electronic components are powered by a battery B, such as a 12 volt or 24 volt battery power source. The indicator 23 and other electrical components are connected to battery B via cables such as cables 15 and 21 with connectors 17 and 19, and connectors 25 and 27 to red and black battery terminals 31 and 33 through a fuse 29. In actual implementation, the indicator 23 may be a visual or audible indicator, and may be placed on or near the fuel filter unit, or may be placed close to the vehicle user, such as at a helm position on marine vehicle.

The incorporation of the indicators 23 allows the user to not have to constantly monitor the fuel filter unit itself, and alerts the user when contaminant in the fuel has reached a threshold level and requires draining.

The sensor 45 and control board 71 may also include a self-test feature which serves to function to inform the user that the system and its audible or visual indicators are operational, and may derive from software designed and installed on the control board 71 having a microprocessor thereon. The self-test may operate upon initial start up of the engine to show that the sensor system and indicator 23 is working.

As may be appreciated, upon detection of water or other contaminants in the system, the visual indicator or audible indicator 23 will serve to alarm the user. The vent for the filter unit 13 (not shown) can be open and drain plug 51 can be removed from opening 49 to allow water to drain out.

As an example of use of the invention, the detection apparatus 10 previously described may be placed between a fuel source and the engine. The user then programs the contaminant detection sensor probe 45 and control board 71 with a predetermined level of contaminant desired for generation of an alarm signal. For example, the sensor may be programmed to generate an alarm signal when the concentration of water reaches 20%. The predetermined level may vary with engine size or type as will be readily apparent to those of ordinary skill in the art.

Once the contaminant threshold has been programmed, fuel may be caused to enter the fuel filter 13, typically by starting the engine. As fuel flows through the system, the detection probe 45 monitors the accumulation of contaminant in the contaminant collection zone or the lower unit 43. It should be noted that since the probe 45 is located towards the bottom of the filter unit 13, because of the relative difference in density between fuel and water, that it is likely that water will be found in a lower region in increasing concentration towards the lower unit 43 thus providing for earlier detection.

Once the predetermined level is reached, the sensor probe 45 and control board 71 generate a signal which is then received by the indicators to alert the user.

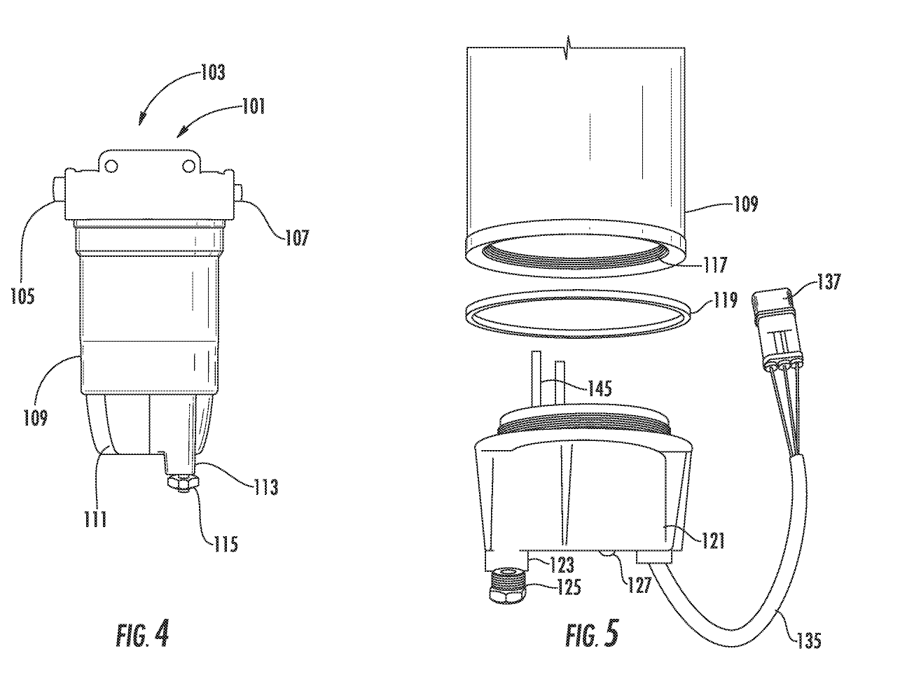

FIG. 4 illustrates an alternative arrangement 101 of a conventional fuel filter. A top section 103 includes a fuel inlet 105, a fuel outlet 107 and a vent (not shown). The top section is designed to attach to a self contained filter unit 109, encased, for example, by a metal shell. The attachment may be achieved by threaded engagement or other like engagement. At the bottom is a containment collection bowl 111, with a discharge opening 113 for containment when a plug 115 thereof is removed.

In accordance with an alternative embodiment of the invention, the prior art device of FIG. 4 may be modified as shown in FIG. 5. More specifically, the self contained filter unit 109 has threads 117 for typically engaging with O-ring 119 interposed, a sensor containing collector bowl 121 containing the control board and contaminant collection zone (both not shown) therein, as described hereinabove. Detection probes 145 extend up and are integral with the sensor containing collector bowl 121. A discharge opening 123 with plug 125 allows discharge of contaminant. An indicator light 127 may be affixed to the system and powered through cable 135 and connector 137 by a battery.

The various components of the invention may be made of standard materials such as metals, plastics, composites or other like materials.

It will be understood that various details of the present invention may be changed without departing from the scope of the present invention. Various aspects of each embodiment described herein may be interchanged with various aspects of other embodiments. Furthermore, the foregoing description is for the purpose of illustration only, and not for the purpose of limitation.

What is claimed is:

1. An apparatus for detecting contaminant in a fuel system, the apparatus comprising:
(a) a fuel filter unit comprising:
(i) an upper unit having an upper section with a fuel inlet port, a fuel outlet port, and a closeable vent port, said upper unit having a lower section with open interior space bounded by walls thereof for receiving a fuel filter, and housing a fuel filter therein; and
(ii) a lower unit releasably mounted to the upper unit at the opening at the bottom of the upper unit for sealing the upper unit, said lower unit having a programmable contaminant detection sensor comprising at least one detection probe extending upwardly therethrough into a fuel compartment region, for generation of a signal upon contact with a predetermined level of contaminant, and a control board within the lower unit connected to the at least one detection probe, and capable of varying the predetermined level; and
(b) at least one indicator for generating an alarm condition in response to receiving the signal.

2. The apparatus of claim 1, further comprising an isolated compartment within the lower unit housing the control board therein.

3. The apparatus of claim 2, wherein the control board includes a programmable microprocessor capable of varying the predetermined level in accordance with programming thereof, and said at least one detection probe and control board together making up a programmable sensor.

4. The apparatus of claim 1, wherein the lower unit further comprises a contaminant collection zone with a sealable opening therein for allowing contaminant to be released through said opening, and further comprising a plug for sealing said opening.

5. The apparatus of claim 1, wherein the contaminant detection sensor is capable of detecting water as the contaminant.

6. The apparatus of claim 1, wherein the programmable contaminant detection sensor is programmed for detecting a level of contaminant comprising 20% water.

7. The apparatus of claim 1, wherein the at least one indicator comprises a visual indicator.

8. The apparatus of claim 7, further comprising a power supply connected to said visual indicator and to said programmable sensor.

9. The apparatus of claim 8, wherein the power supply is a battery.

10. The apparatus of claim 1, wherein the programmable sensor is formed integrally within the lower unit.

11. The apparatus of claim 1, wherein the at least one indicator comprises an audible indicator capable of generating an audible alarm condition.

12. The apparatus of claim 11, further comprising a power supply connected to said audible indicator and to said programmable sensor.

13. The apparatus of claim 1, further comprising a fuel system having the apparatus connected therein combined with an internal combustion engine.

14. The apparatus of claim 13, wherein the fuel system and internal combustion engine are a marine fuel system and marine internal combustion engine.

15. The apparatus of claim 1 wherein said upper section of said upper unit is releasable therefrom.

16. An apparatus for detecting water in a fuel system of an internal combustion engine, the apparatus comprising:
(a) a fuel filter unit comprising:
(i) an upper unit having an upper section with a fuel inlet port, a fuel outlet port, and a closeable vent port, said upper unit having a lower section with an open interior space bounded by walls thereof for receiving a fuel filter and housing a fuel filter therein, and having an opening at the bottom thereof; and
(ii) a lower unit releaseably mounted to the upper unit at the opening at the bottom of the upper unit, said lower unit having a programmable water detection sensor comprising at least one detector probe extending upwardly therethrough into a fuel compartment region, for generation of a signal upon contact with a predetermined level of water, an isolated compartment within the lower unit housing a control board with a programmable microprocessor thereon capable of varying the predetermined level in accordance with programming thereof, and the control board connected to the at least one detector probe; and
(b) at least one indicator for generating an alarm condition in response to receiving the signal.

17. The apparatus of claim 16, further comprising a water collection zone with a sealable opening therein for allowing water to be released through the opening, and further comprising a plug for sealing the opening.

18. The apparatus of claim 17, further comprising a fuel system with an internal combustion engine connected thereto, and having the apparatus connected within the fuel system.

19. A method of detecting contaminant in a fuel system of an internal combustion engine, the method comprising:
(a) providing a contaminant detection apparatus comprising:
(i) a fuel filter unit comprising an upper unit having the upper section with a fuel inlet port, a fuel outlet port and a closeable vent port, said upper unit having a lower section with an open interior space bounded by walls thereof for receiving a fuel filter and housing a fuel filter therein, and having an opening at the bottom, (ii) a lower unit releasably mounted to the upper unit at the opening at the bottom of the upper unit for sealing the upper unit, said lower unit having a programmable contaminant detection sensor comprising at least one detection probe extending upwardly therethrough into a fuel compartment region, for generation of a signal upon contact with a predetermined level of contaminant, and a control board with a programmable microprocessor thereon housed within the lower unit, and connected to the at least one detection probe and capable of varying the predetermined level, and (iii) at least one indicator for generating an alarm condition in response to receiving the signal; and
(b) placing the contaminant detection system between a fuel source and an internal combustion engine;
(c) programming the microprocessor with a predetermined level of contaminant selected for generating the alarm signal;
(d) causing fuel to enter the contaminant detection apparatus from a fuel source; and
(e) monitoring the fuel and generating the alarm signal when the predetermined level of contaminant is reached.

20. The method of claim 19, further comprising generating at least one of a visual and audible signal with said indicator.
